(19) United States
(12) Patent Application Publication
(10) Pub. No.: US 2008/0013678 A1
(43) Pub. Date: Jan. 17, 2008

(54) Device for Placing Instruments or Implants in Body Organs

(75) Inventors: Friedrich Magerl, St. Gallen (CH); Christian Widmer, St. Margrethen (CH); Stanislav Taller, Liberc (CZ); Richard Lukas, Liberec (CZ); Petr Suchomel, Liberec (CZ)

Correspondence Address: BROWDY AND NEIMARK, P.L.L.C.;624 NINTH STREET, NW, SUITE 300, WASHINGTON, DC, 20001-5303, US

(73) Assignee: SEPITEC FOUNDATION, Vaduz (LI)

(21) Appl. No.: 10/573,761

(22) PCT Filed: Sep. 22, 2004
(86) PCT No.: PCT/CH04/00596

§ 371 Date: Feb. 16, 2007

(30) Foreign Application Priority Data
Sep. 29, 2003 (CH) 1651/03

(51) Int. Cl. A61B 6/12

(52) U.S. Cl. 378/20

(57) ABSTRACT

A device for precisely placing instruments or implants in body organs with the aid of a targeting device (40) interacting with a computer tomograph, characterized in that the targeting device is arranged in the form of a navigation device (30) on a carrier (2) which is diplaceable on a substructure like a carriage which can be fixed in relation to the substructure and in that the navigation unit (30) comprises a retaining element for holding and guiding a targeting instrument, implant or other object. The navigation unit (30) can be displaced and/or rotated on one or several planes in relation to the carrier and the retaining element can be displaced and/or rotated on one or several planes in relation to the navigation unit (30), being reciprocally fixable in the adjusted position thereof.

The invention relates to a device for percutaneous placing of instruments or implants in body organs in accordance with the preamble of claim 1, and to a method for operating such a device.

In seeking to reduce the iatrogenic trauma of surgical interventions, minimally invasive and percutaneous operating techniques are increasingly being used in surgery. Because of the relatively high level of safety of CT navigation, a CT targeting appliance widens the range of indications for these operating techniques. Precision and safety are of the utmost importance in a number of percutaneous or minimally invasive interventions: on the spinal column, for example for placing screws, pins or cannulas (e.g. pedicle screws, translaminar pin fixation, biopsies, vertebroplasty); on other areas of the skeleton, for example the pelvis, where the use of conventional navigation techniques may cause considerable difficulties because of the complex anatomical circumstances; and generally in cases where a high degree of targeting accuracy is needed, for example for biopsies of small pathological foci or tumor nests. CT navigation, which does not require a high technical outlay, is more precise, more reliable and simpler to handle and is considerably less expensive than computer-assisted navigation techniques.

In a minimally invasive operation, the target object (e.g. a herniated disk) is made visible through a small open access route. Viewing aids such as microscopes, or endoscopes equipped with cameras, and special instruments and navigation aids are technical prerequisites for such interventions. Percutaneous interventions are carried out without an open access through the tissues covering the target object, and therefore without direct viewing of the target object. The target object is represented with the aid of an imaging method (e.g. X-ray image converter, ultrasonography, computed tomography, magnetic resonance imaging), and instruments and implants are inserted into the body with the aid of these methods and/or special navigation aids (e.g. computer-assisted surgery (CAS)).

Computer-assisted surgery (CAS) has acquired particular importance. In the traditional method, the intervention is planned on the basis of computer tomographs obtained prior to surgery. In the operating theater, the virtual target object, produced beforehand by computed tomography, is brought into congruence with the real object, using a stereophotogrammetric camera and special instruments, so that, finally, the coordinates of the two objects exactly match. This procedure is called referencing. To ensure that the match is preserved even in the event of possible changes of position of the target object, an instrument (reference base) has to be secured on this and transmits positional changes to the computer. Thereafter, surgery can be performed with standardized instruments equipped with markers or diodes. The camera and computer determine the spatial position of the instruments. Their relationship to the target object (e.g. a region of the spinal column) is in each case depicted online on the screen. Weaknesses of these techniques are the laborious and often difficult referencing and the fact that percutaneous interventions are not possible because of the requirement for referencing.

In image-converter-assisted navigation (3D C-arm navigation), there is no reliance on computer tomographs obtained prior to surgery. A pre-calibrated navigated image converter (IC) is implemented in the system. After the reference base has been secured on the patient, a series of IC images is recorded, loaded into the navigation computer and processed by the latter. Surgery can thereafter be performed with calibrated instruments. The target object and instruments are viewed on the screen. Changes in the position of the target object or the position of the inserted instruments and implants can be viewed during the operation by creating new IC images for continued navigation. Weaknesses of IC navigation are the image converter's limited resolution, inadequate for interventions on the cervical spine and thoracic spine, and the need for a reference base. The latter once again makes it impossible to carry out purely percutaneous interventions.

Safe operation of the stated systems requires special training of the operating surgeons. The availability of instruments is limited to calibrated instruments and ones equipped with markers, and the use of flexible and long, thin instruments, for example long drills or cannulas, can cause considerable problems. This, and the high costs of the CAS systems, limits the use of these systems to interventions which are also complicated under traditional operating conditions.

A device and a method for intraoperative navigation have been disclosed in WO 02/062250 A1. To place a medical implant, a mobile medical imaging appliance is used which has a position-recording device for spatial position measurement of reference elements relative to a spatially fixed coordinates system. A reference element is secured in each case on the imaging unit, on the bone to be treated, and on the surgical instrument. At least one radiation source and at least one receiver unit are connected to the spatially movable imaging unit, and a further reference element is secured on this imaging unit. The device and the method are intended to be based on a system of reference coordinates formed by only a few anatomical landmarks. The referencing required here is prone to failure and is relatively difficult. It is not possible for purely percutaneous interventions to be performed by the technique described in said patent specification.

A method for implantation of a knee prosthesis by means of computer-assisted navigation has been disclosed in U.S. Pat. No. 5,682,886. By means of a computer tomograph, a three-dimensional computer model of the body part in question is created. Complex referencing, which is prone to failure, is needed in this case too. Purely percutaneous interventions are not possible using the technique described in said patent specification.

U.S. Pat. No. 6,221,082 has disclosed a device which, on a fixed platform on a horizontal support, has two displaceably mounted cannula supports. These two cannula supports are guided such that the cannulas remain coplanar to one another when the cannula supports are moved to different positions in an intervertebral plane of a patient. Radiographic calibrated markers are secured on the cannula supports and are replaced by cannulas after images have been recorded. The cannulas are used for introducing surgical objects into the intervertebral space of the patient.

In this case, image intensifiers are used as imaging medium for introducing instruments or implants into the vertebral column. The resolution of image intensifiers is known to be limited, especially when compared to computed tomography.. Only osseous structures can be represented with the image intensifier. Soft structures important for safe navigation in the area of the vertebral column, for example the spinal cord or nerve roots, are not shown. Strong superimposition of the osseous elements by thick soft parts, as is the case in adipose patients, can also greatly impair the view of osseous elements of the vertebrae. The technique described here is unreliable and of only limited application.

The object of the present invention is therefore to make the use of image-assisted navigation economically feasible in minor interventions too, and in particular to permit percutaneous interventions.

The object is achieved with a device in accordance with claim 1.

The technical effects and special advantages of the invention are explained in greater detail in the description that follows. Further features are set forth in the dependent claims too. An important advantage of the invention is seen in the fact that it can be used for percutaneous interventions in numerous regions of the skeleton. However, it is additionally suitable also for percutaneous interventions on soft parts, except those which, for example like the heart and lungs, are in constant movement in order to maintain vital functions. It is also important that, in percutaneous interventions, the invention guarantees the necessary precision and safety and does so without great technical outlay and increased costs.

According to the invention, the CT navigation under discussion is therefore achieved with the aid of a targeting appliance that interacts with the computer tomograph. This targeting appliance consists of a base plate and of the structure mounted thereon. This structure consists of at least one straight support column which is preferably vertical with respect to the base plate. If two support columns are provided and they are connected at the upper ends or near their upper ends by a straight crossbeam, the unit made up of support columns and crossbeam forms a gantry-like frame (gantry). Devices that are displaceable on base rails, and ensure exact guiding, connect the support columns to the base plate. At least one navigation unit is preferably secured in each case on the crossbeam or the support columns. This navigation unit consists of a cylinder retainer, the inclinometer, and a cylinder with several exchangeable and combinable sleeves, which form a guide, and instruments.

In computer-assisted navigation, at least the referencing to the data stored in the computer (virtual object) is indispensable. Since the body has to be opened for this purpose, the application of a purely percutaneous navigation technique is not possible. In the device according to the invention, navigation is carried out not with the aid of a virtual object, but on the basis of updatable CT images. Thus, without switching between potential sources of error such as reconstruction and referencing, it is possible to ensure certainty regarding the situation in question. A departure from the operation plan can be immediately identified and corrected. This, and the high degree of precision, gives the device according to the invention a hitherto unattainable level of safety and opens up in particular the possibility of navigated percutaneous interventions.

In computed tomography, series of transverse or oblique sections of the body part in question are computer-generated with the aid of an X-ray tube circulating rapidly in the gantry of the tomograph, around the patient's body part lying in the gantry opening, and are viewed on the screen and shown in printed-out section images. The resolution of computed tomography is very high, and it is in particular much higher than that of image intensifiers. Whereas the image intensifier is suitable only for showing osseous structures, computed tomography also shows soft-tissue structures such as intervertebral disks, nerves, ligaments, etc. However, the computed tomography representation of objects and organs is not limited to these easily generated section images. By computer processing of the image data, it is possible to generate a) three-dimensional views of the recorded organs and b) also section images in each desired plane. Since the computing process required for this purpose takes a certain amount of time, and because these views are not needed for the moment, only the CT navigation based on directly obtained section images will be discussed below. It should be noted, however, that the additional possibilities cited under a) and b) in this paragraph can also be exploited if necessary for CT navigation. This possibility will be made use of if the implant/instrument to be introduced does not lie in a section plane or if, for whatever reasons, visualization with particularly high resolution of the target object and/or of the introduced object is desired.

Particularly suitable for simple CT navigations that can be carried out in one section plane are all objects which can be imaged in one section plane and which are radiologically sufficiently viewable, without causing disruptive artefacts, and can be introduced along a straight line (insertion or implantation axis) toward the target object. To satisfy the last-mentioned condition, the objects should, in the straight line (object axis) marking their insertion axis, have at least two sufficiently precisely definable orientation points. Accordingly, suitable objects for simple CT navigation are chiefly objects which are long and are to be introduced in their longitudinal direction, for example pins, needles, screws or cannulas.

An illustrative embodiment of the invention is explained in more detail below with reference to the drawing.

For further explanation of the features according to the invention, the device variants are shown in partially schematic representations in the drawings.

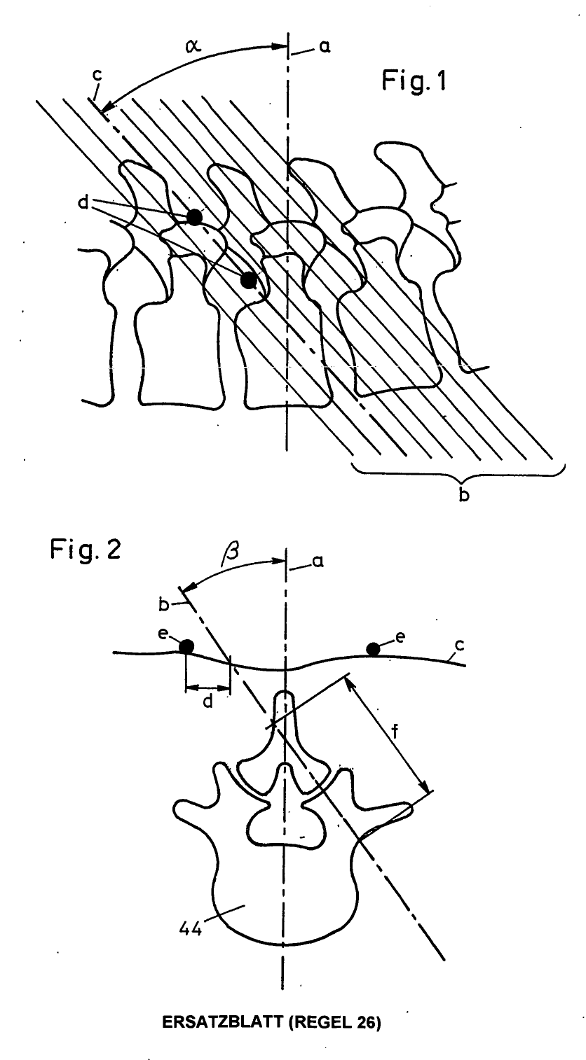

FIG. 1 shows a CT navigation, taking as an example a translaminar facet screw connection on the lumbar vertebral column, a lateral scout image being shown with the section planes;

FIG. 2 shows a view of the selected section image (work plane);

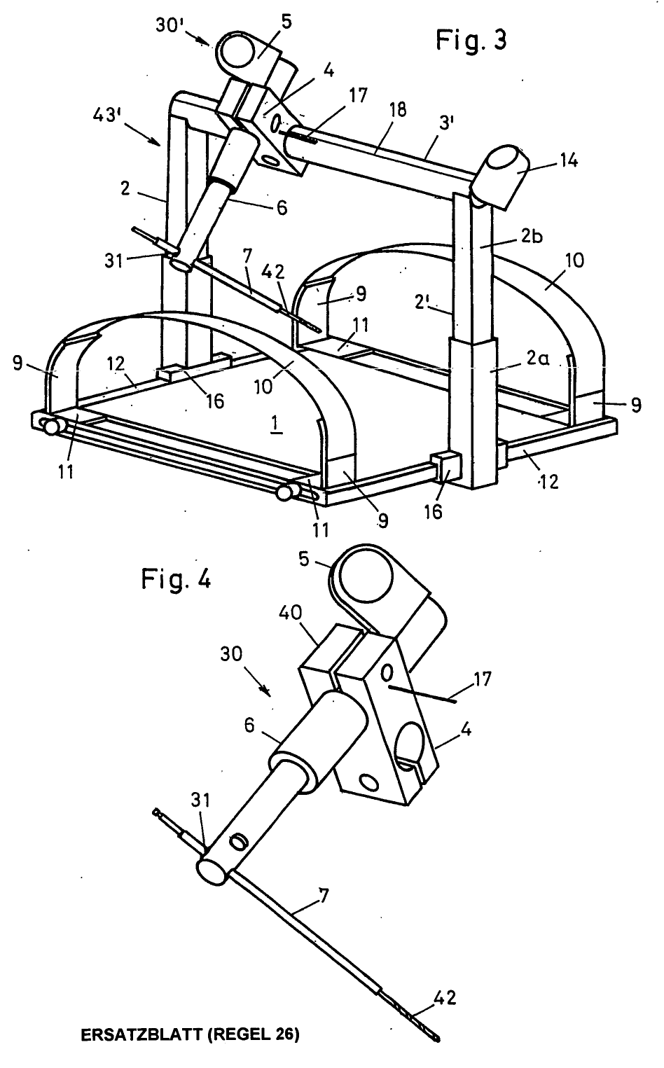

FIG. 3 shows a CT targeting appliance without devices for securing the targeting appliance on the CT table;

FIG. 4 shows a navigation unit of the CT targeting appliance;

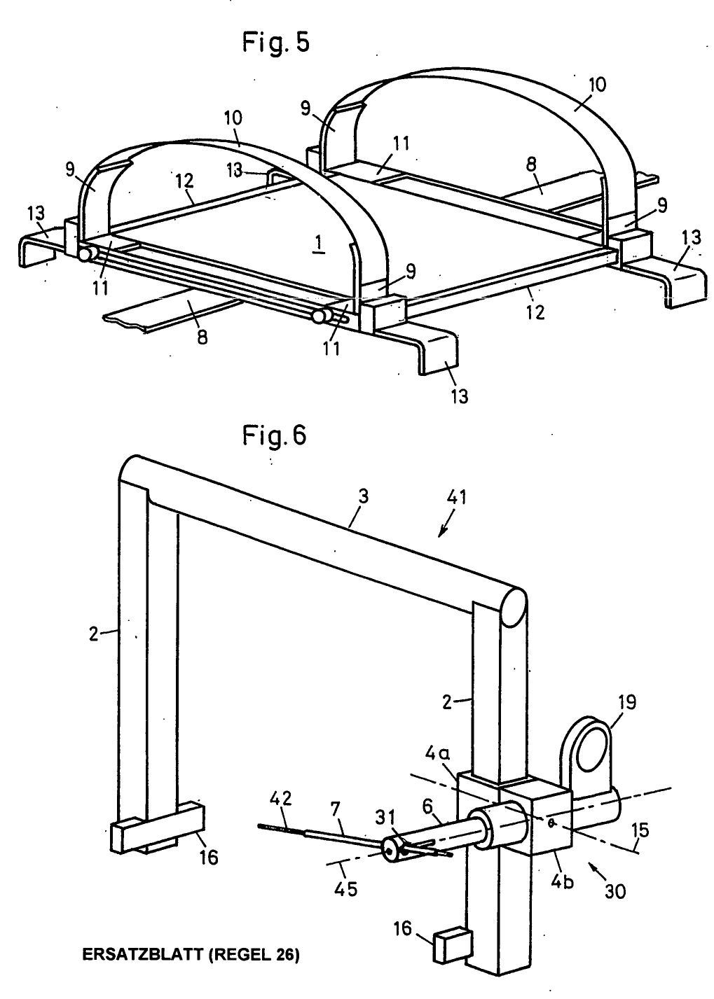

FIG. 5 shows a base plate with retainers;

FIG. 6 shows a gantry-like support, with a navigation unit fitted on a vertical support column;

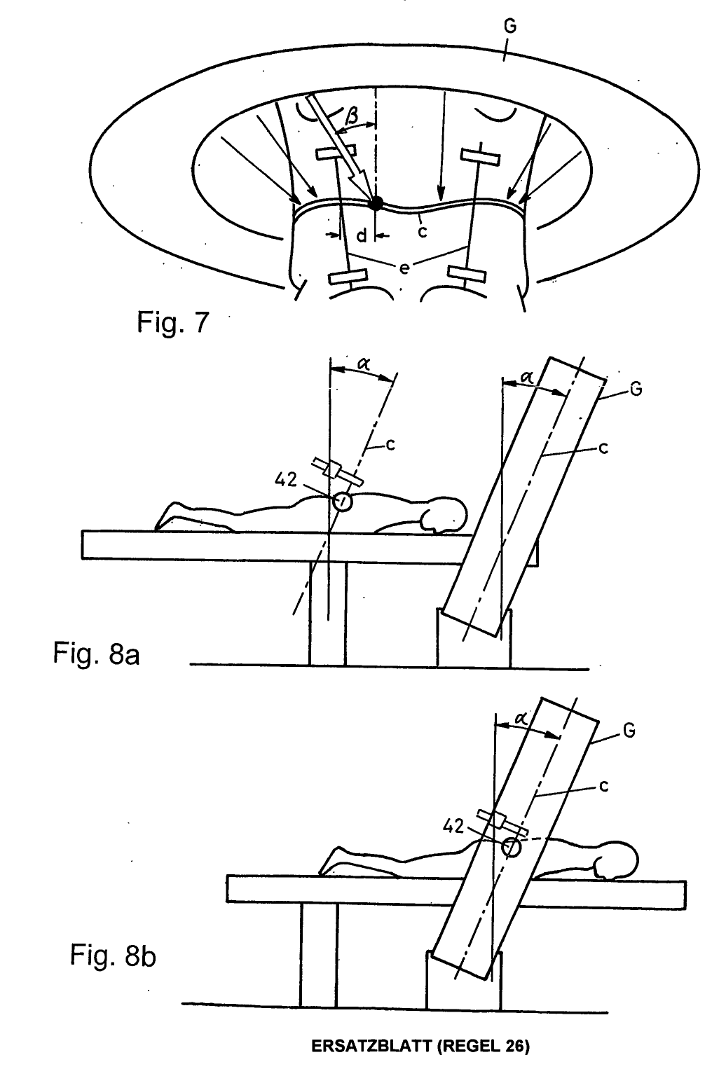

FIG. 7 shows a schematic representation of the CT navigation principle according to the invention;

FIGS. 8a+8b show schematic representations of the sequence of the CT navigation according to the invention;

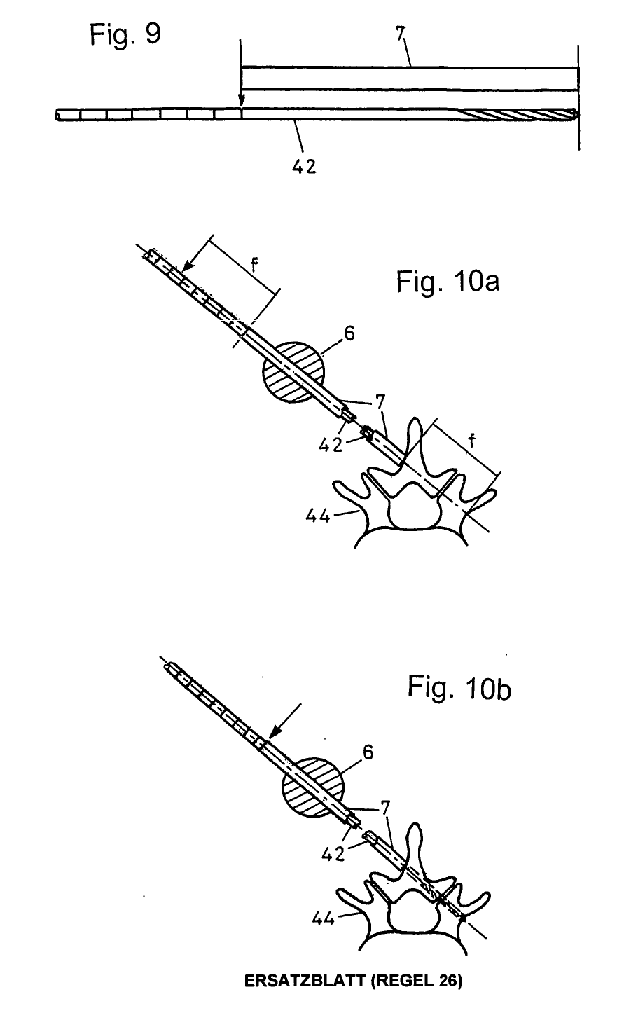

FIG. 9 shows a schematic representation of a guide sleeve with matching instrument;

FIGS. 10a+10b show an example of how the depth of penetration of an instrument or implant is measured.

To perform CT navigation by means of the device according to the invention, a series of test sections is needed from which the appropriate section plane (work plane) can be chosen, as is shown in FIG. 1. For this purpose, a scout image of the intended target region is first prepared with the CT. Using anatomical features, and with the aid of the scout image, the tilt angle α of the test sections with respect to the horizontal is fixed, the target region for the test sections is demarcated, and the spacings of the test sections relative to one another are determined. To prepare the test sections, the gantry of the computer tomograph (not shown here) has to be tilted about the angle α. When preparing the test sections, the movable part of the CT table with the patient is automatically moved into the region of the test sections and advanced by the predetermined spacings of the section planes. The position adopted in each case by the CT table is stored in the CT. The test sections are each shown on the screen. The position of the instruments/implants to be inserted, hereinafter referred to as objects, is then plotted in the appropriate section plane (work plane) into the section image with a cursor, as is shown in FIG. 2. This yields the insertion angle β (insertion axis) lying in the section plane with respect to the vertical likewise lying in the section plane. In the section image, the depth of penetration and length of the objects are also determined with the cursor. The angles α and β and the depth of penetration and lengths of the objects are calculated by the CT computer and displayed in the data block of the section image on the screen and also on printed-out section images. The tilt angle α is also indicated on the gantry. The procedure described here is represented schematically in FIGS. 1, 2 and 7.

The actual principle of CT navigation involves, after these preparations, the object to be introduced being brought into the work plane with the aid of the targeting appliance in such a way that it is situated completely therein and can be further moved only in this plane. The object is thereafter displaced in the work plane and/or rotated about an axis perpendicular to the work plane until its object axis lies exactly in the insertion axis previously defined in the section image. As soon as this has happened, the setting of the targeting appliance is fixed in such a way that the object can now be displaced only along the insertion axis, i.e. toward the target organ or away from it.

The CT navigation is preferably effected with the aid of a targeting device which interacts with the computer tomograph and which is shown in FIG. 3. It consists of a base plate 1 and of a targeting appliance 40 mounted thereon. The targeting appliance has at least one straight support column 2 preferably vertical with respect to the base plate 1. If two support columns 2 are provided and these are connected at the upper ends or near their upper ends by a straight crossbeam 3, the unit made up of support columns and crossbeam forms a gantry-like frame (gantry). Devices 16 that are displaceable on base rails 12, and ensure exact guiding, connect the support columns 2 to the base plate 1. At least one navigation unit 30 is preferably secured in each case on the crossbeam 3 or the support columns 2. This navigation unit 30 consists of a cylinder retainer 4, of an inclinometer 5, and of a cylinder 6 with several exchangeable and combinable retaining elements in the form of sleeves, which form a guide, and instruments 7. Bores 31 permitting exact guiding are provided for the inclinometer in the cylinder.

For insertion axes inclined mainly to the vertical, at least one navigation unit 30 is secured on the crossbeam 3, as is shown in FIG. 3. For insertion axes inclined mainly to the horizontal, the navigation unit is preferably arranged on a vertical support column 2. For insertion axes inclined mainly to the horizontal, it would be conceivable to use a support column 2, with navigation unit 30, only on one side.

The patient lies on the rectangular base plate 1 that allows X-rays to pass through. The base plate 1 is narrower than the longitudinally displaceable part of the CT table and long enough to ensure that the targeting appliance has the displaceability necessary for the operation. The base plate moves with the displaceable part of the CT table into the gantry opening and out of it (FIGS. 8a, 8b). To avoid undesired changes of position of the targeting appliance, retaining devices are provided which secure the base plate 1, the body of the patient and/or the target organ itself. With belts 8, the base plate 1 is secured on the movable part of the CT table such that it cannot slip on this, but does not obstruct the mobility of the table. The body of the patient is fixed on the base plate 1 via lateral struts 9 and by belts 10 that are stretched transversely across the patient's body. The devices 11 connecting the struts 9 to the base rails 11 can also be configured in such a way that the struts 9 are displaceable thereon transverse to the longitudinal axis of the body and the retaining means can thus be adapted to the width of the patient's body. If the target organ is an osseous structure, it can be fixed by one or more pins or screws being fitted into the bone near the target region and by these being secured on the gantry or base plate 1 with retaining devices.

The base rails 12 for securing the support columns 2 are provided on lateral edges of the base plate 1. On these rails 12, the targeting appliance 40 can be displaced in the longitudinal direction of the CT table. Devices 13 which secure the base plate 1 on the displaceable part of the CT table can also be fitted on the base rails 12.

The gantry 41 consists of two straight support columns 2 which are secured preferably vertically on the base rails 12 and which are connected by the horizontal, straight crossbeam 3. The gantry 41 is displaceable on the base rails 12 in the longitudinal direction of the CT table such that the horizontal crossbeam 3 remains parallel to the gantry plane or work plane. If the two support columns 2 consist of in each case two parts 2a and 2b which are displaceable one within the other in the manner of a telescope, the height of the gantry can be adjusted. It would also be possible to make the height of the gantry adjustable by securing the crossbeam 3 to the support columns 2 by suitable devices in such a way that it can be displaced up and down along these in parallel.

It would also be conceivable to make the width of the gantry 41 adaptable to the dimensions of the patient. To do so, displaceable devices could be fitted on the base rails 12, on which devices the vertical support columns 2 are displaceable not only along the base rails 12, but also perpendicular to the base rails 12. In this case, the length of the crossbeam 3 would also have to be adaptable to the gantry width. For this purpose, the crossbeam 3 could likewise consist of two parts 2a and 2b displaceable one within the other in the manner of a telescope. However, it would also be conceivable to use a crossbeam 3 whose length is sufficient for the greatest possible adjustable width of the gantry. The devices connecting the crossbeam 3 to the support columns 2 would in this case also have to permit, in addition to possible rotation of the crossbeam 3, the latter's displaceability in its longitudinal axis. For the adjustment of the gantry width, it would suffice if the crossbeam 3 were displaced in the stated sense only in one of the devices.

The crossbeam 3 carries at least one navigation unit 30 that can be moved along it. The navigation unit 30 is preferably rotatable perpendicular to the longitudinal axis of the crossbeam 3. To ensure this rotatability, the crossbeam 3 can be secured rotatably on the support columns 2 and the navigation unit 30 can be secured only displaceably on the crossbeam 3, such that the navigation unit 30 has to turn together with the crossbeam 3. To ensure that the navigation unit 30 can turn, it can be designed such that it can rotate about a crossbeam 3 of round cross section (FIG. 6), or the crossbeam can be connected in terms of rotation about its longitudinal axis to the support columns 2. The navigation unit 30 then turns together with the crossbeam. The inclinometer 5 measuring the rotation angle α could then be arranged both on the navigation unit 30 and also on the crossbeam 3. Another possibility is to secure the navigation unit 30 on the crossbeam 3 such that it can both be displaced along the crossbeam 3 and also rotated about the crossbeam 3. In this case, the crossbeam 3 is not connected rotatably to the support columns 2, and the cylinder retainer 4 has to be provided with a device that permits its rotation about the crossbeam 3. In this variant, the navigation unit 30 is rotatable about the longitudinal axis of the crossbeam 3.

For insertion axes inclined mainly toward the horizontal, the navigation unit 30a is fitted on a vertical support column 2 according to FIG. 6 in such a way that it can be moved only up and down on the support column 2 and cannot be rotated about the longitudinal axis of the support column 2. To ensure that the navigation unit 30 can be tilted according to the angle α, a uniaxial hinge shown in FIG. 6 is arranged between the part 4a of the cylinder retainer 4, which connects this to the support column 2, and a rotatable part 4b. The axis 15 of the hinge extends parallel to the present or only imaginary crossbeam 3, in any case parallel to the tilt axis of the gantry. This thus ensures that the angle α can be adjusted by tilting the part 4b about the axis 15.

The cylinder 6 mounted in the cylinder retainer 4 and not casting a shadow is designed exactly as has been described above and is movable in the cylinder retainer 4. The cylinder 6 does not form a shadow in radiology terms and thus allows X-rays to pass through. The work bore 31 of the cylinder 6 preferably extends at right angles to the longitudinal axis of the cylinder 6. Since the cylinder retainer 4 can be tilted about the angle α, this ensures that the object to be introduced can be brought into the work plane or insertion axis by advancing and rotating of the cylinder 6. The inclinometer 19 is arranged on that end of the cylinder 6 remote from the work bore 31, such that it can be turned through 90 degrees in the bore provided for it. The angle α is measured with the inclinometer set parallel to the longitudinal axis of the cylinder, and the angle β is measured with it turned through 90 degrees.

A design is also conceivable in which several cylinders 6 with different diameters and different cross-sectional shapes of the work bore 31 are provided. The instruments/implants 42 are introduced into the body through the sleeves 7 fitting in each case into the work bore 31. The shape and sizes of the sleeves 7 are such that the sleeves 7 exactly match the instruments/implants 42 being used and guide them exactly. The sleeves 7 can be made of metal or of a material that allows X-rays to pass through.

The targeting device 43 has several displaceable or rotatable parts. Thus, the support columns 2 can be moved along the base rails 12 such that the crossbeam 3 always moves parallel to the axis of the gantry tilt. The support columns 2 can consist of parts 2a, 2b which are displaceable one within the other in the manner of a telescope. The crossbeam 3 can be secured on the support columns 2 both in a fixed state and also in a displaceable and/or rotatable manner. The cylinder retainer 4 can be arranged only displaceably on the crossbeam 3, or in addition also rotatable about the latter. The cylinder 6 mounted in the cylinder retainer 4 is mounted displaceably and rotatably in the cylinder retainer 4. A navigation unit 30 mounted on the support column 2 can be moved along the latter and is designed such that rotation is possible about an axis which is parallel to the axis of the gantry tilt.

The mobility of the targeting device 43 and therefore also of the navigation unit 30 ensures the adjustability of the implant axis of the instruments/implants 42 with respect to the target organ 44. The parts can be adjusted directly by hand. Mechanical precision drives that can be operated by hand or are operated by motor are advantageous. For all the movable parts of the targeting device 43, devices (not shown here) are also provided with which the mobility of these parts can be blocked at any time.

For measuring the angles α and β, high-precision and preferably digital inclinometers with built-in spirit level 19 are provided which are either mounted in a fixed position or are removable. If the crossbeam 3 is mounted rotatably on the support columns 2, the inclinometer 5 for the angle α can be secured both on the navigation unit 30 and also on the crossbeam 3. If, by contrast, the crossbeam 3 is not rotatably connected to the support columns 2, the inclinometer 5 for the angle α has to be secured on the navigation unit 30. The inclinometer 5 for the angle β is mounted on the cylinder 6 itself, preferably near that end of the cylinder 6 lying remote from the work bore 31, as is shown in FIGS. 3, 4 and 6. The cylinder bore for the inclinometer is designed such that the inclinometer can only be mounted exactly parallel to the longitudinal axis of the cylinder or perpendicular thereto.

One possible way of controlling the tilt angle α is if a laser light is emitted from the gantry exactly in the section plane of the computer tomograph, and this laser light marks the respective section plane. The tilt angle α can therefore be controlled by a pointer 17 being mounted on the cylinder retainer 4 above the crossbeam 3 and oriented parallel to the crossbeam 3, and by a line 18 being arranged on the crossbeam 3 such that the shadow of the pointer 17 generated by the laser light then impinges on a line 18 when the tilt of the cylinder retainer 4 corresponds to the gantry tilt and thus to the angle α. The following possibility of controlling the angle α would also be conceivable: After the work plane is determined, its section plane with the body surface is marked on the skin of the patient with the aid of said laser light. When, after adjusting the navigation unit 30 to the angle α, a targeting instrument is fitted into the work bore 31 and this is advanced in the cylinder retainer 4 until its tip touches the indicated section line, the laser light has to fall exactly on the center of the work bore 31 when the gantry is adjusted to the work plane. The adjustment can be more easily controlled with the aid of a marking extending transversely across the cylinder from the center of the work bore.

The described navigation principle is achieved by the navigation unit 30 being tilted about the angle α. By advancing the targeting appliance on the base rails 12 and/or the cylinder 6 in its retainer, the object to be introduced can thereafter be brought into the work plane. A precondition for this is that the cylinder 6 encloses a right angle with the work plane and that the object 42 to be introduced encloses a right angle with the cylinder 6. In this arrangement, the axis of the object 42 to be introduced extends in each case parallel to the work plane, such that the object 42 can be pushed into the work plane. By moving the navigation unit 30 on the crossbeam 3 or on the support column 2, and by turning the cylinder 6 in its retainer, the axis of the object 42 to be introduced can then be brought exactly into the predetermined insertion axis. The course of the operation/navigation is controlled using recurrently prepared CT sections.

The objects 42 mentioned are instruments, e.g. drills, screwdrivers or cannulas, and implants, e.g. pins, screws. The external diameters of the objects and the internal diameters of the associated guide sleeves 7 are in each case adapted to one another such that the objects are inserted into the sleeves with a form fit but without jamming.

For performing a biopsy and for introducing an implant, it is important that these objects can be positioned in the target organ 44 exactly as intended (cf. FIG. 2). The implant position and implant length f, as shown in FIG. 2, are plotted with the cursor in the section image of the work plane, determined by the computer of the CT appliance, and displayed in the data block of the section image. For introduction of an implant, the distant end of the implant in the insertion direction b at the same time determines its depth of penetration into the target organ. The same depth of penetration applies to instruments, e.g. the drill, with which the channel for the implant is created. To ensure that the objects can be brought exactly into the position determined in the section image, special steps are needed. The instruments needed for the implantation are to be provided with a measurement scale whose zero mark lies exactly where the rear end of the matching guide sleeve is situated when the lengths of the sleeve and of that part of the instrument lying in front of the zero mark are identical (FIG. 9). Moreover, it is necessary to define, in the section image, the position of the more remote end of the guide sleeve 7 (front sleeve end) in the insertion axis. When this sleeve end, as shown in FIG. 10, lies on the target organ, it suffices to introduce the instrument or implant by the length f in order to bring it into the intended position. If the front sleeve end is situated at some distance from the target organ, then the distance of the front sleeve end from the nearer end of the implant position f (FIG. 2) has to be added to the implant length f so that, for example, the drill penetrates deep enough and the implant can be brought into the intended position.

The guide sleeves and biopsy instruments provided for performing a biopsy are configured just like the above-described guide sleeves and instruments. In addition, the biopsy instruments are also equipped with a measurement scale.

In the case of a biopsy, the distance of the front sleeve end from that site of the target object (e.g. abscess or tumor). from which the biopsy specimen is to be taken is measured in the section image of the work plane. With the aid of the measurement scale, the tip of the instrument can then be brought to the site in question.

During the -above-described procedure of inserting an instrument or implant into the correct position, the position and length of the respective guide sleeve ought not to be changed. Clamping the guide sleeve 7 in the cylinder 6 ensures that the guide sleeve in the cylinder cannot move along the insertion axis b.

In the course of an intervention, the patient and the targeting device 43 are generally moved out of the gantry a number of times with the movable CT table (FIG. 8). If the setting of the targeting appliance 40, the tilt of the gantry and the position of the patient's body are not changed, automatic return of the CT table to the stored position of the work plane allows navigation to continue in this work plane. The reproducibility of this procedure facilitates the CT navigation and makes the course of the operation precisely controllable each time.

A movement of the patient's body during an intervention can cause a shifting of the target organ 44 and thus an incorrect positioning of the object 42 or instrument/implant. Maintaining all the positions adopted prior to preparation of the test sections is thus an important prerequisite for straightforward CT navigation. A shifting of the target organ 44 can also occur in particular in interventions on osseous structures, if an instrument, for example a drill, has to overcome increased resistance. To ensure that the patient cannot actively move, it is recommended that the planned intervention be carried out under general anesthesia. To prevent changes in position, retaining devices are provided which secure the base plate 1, the patient's body and/or the target organ 44 itself. The base plate 1 is first secured on the movable part of the CT table in such a way that it cannot slip on the latter but does not obstruct the mobility of the table. Before the test sections are prepared, the patient's body is fixed on the base plate 1 with lateral struts 9 and with belts 10 stretched transversely across the patient's body. If the target organ 44 is an osseous structure, it can be fixed by one or more pins or screws being fitted into the bone near the target region and by these being secured on the gantry 41 or base plate 1 with retaining devices (not shown here).

The conduct of a navigated percutaneous intervention by means of the device according to the invention is explained in detail below.

With the aid of the CT targeting appliance, a navigated percutaneous intervention proceeds in the following steps:
a. Securing the base plate 1, with the retaining devices (8) provided for this purpose, on the longitudinally displaceable part of the CT table.
b. Placing the patient on the base plate 1 and securing the patient on the base plate 1, e.g. with struts (9) and belts (10).
c. Mounting the targeting device 43 in such a way that it does not disturb the preparation of a scout image (FIG. 1).
d. Introducing the patient into the gantry, preparing the scout image of the target region.
e. Determining the section planes on the basis of the scout image according to FIG. 1. The tilt angle alpha of the gantry is now determined on the basis of determined points d (FIG. 1) definable in the scout image. The tilt angle alpha of the section planes with respect to the horizontal, and thus the gantry tilt, is indicated on the screen and on the gantry.
f. Removing the patient from the gantry, tilting the gantry of the computer tomograph about the angle alpha.
g. Approximately where the instruments 42 are to be introduced into the body, a sufficiently long and thin metal wire (FIG. 7) is affixed in the longitudinal direction onto the patient's skin. If necessary, it is also possible for several wires to be affixed.
h. Introducing the patient into the gantry, preparing a series of test sections b (FIG. 1) through the target region. In the section images b, the wire mentioned under g. appears as metal point e lying on the skin (FIG. 2).
i. Selecting the appropriate section plane as future work plane c (FIGS. 1 and 2) on the basis of the series of section images.
j. Adjusting the gantry to the level of the work plane.
k. With the laser beam of the computer tomograph, the section line of the work plane with the body surface is projected onto the skin of the patient. The section line is drawn on the skin (FIG. 7).
l. With the aid of electronic input instruments, the planned position of the object 42 is plotted as a straight line b (FIG. 2) into the image of the work plane c shown on the screen of the computer tomograph. This straight line forms the implant axis. The inclination of the implant axis in the work plane c with respect to the horizontal or vertical, preferably the vertical, i.e. the angle β (FIG. 2), is indicated on the screen. All the angles and lengths relevant to the intervention can be determined on the screen and read off from the screen, in particular also the depth of penetration of the object 42 and its length f (FIG. 2). All values can thus be exactly determined in advance.
m. The distance d (FIG. 2) of the insertion axis from the affixed wire at skin level is determined in the same way. Starting from the wire, this distance is marked in the plotted section line of the work plane. This therefore defines at which site the objects 42 (e.g. targeting instrument) are to be inserted into the body.
n. Tilting of the cylinder 6 with the cylinder retainer 4 about the tilt angle α. There are three possible ways of adjusting and controlling the tilt: In the first variant, the tilt is adjusted with the inclinometer provided for this purpose. The tilt is then controlled with the aid of the pointer 17 fitted on the cylinder retainer 4 and the laser light. Finally, the tilt is controlled with the aid of the laser light of the gantry adjusted to the section plane. For the second possibility, the CT table has to be moved in the gantry opening until the laser light impinges on the marking line 18 of the crossbeam 3. With a correctly adjusted tilt, the shadow of the pointer 17 fitted on the navigation unit then falls on the marking line 18. The third possibility is explained under item p.
o. By moving the CT table, the gantry tilted about the angle α is adjusted again to the level of the work plane c. The CT table is again pushed, to the level of the work plane, into the gantry that has been tilted about the angle α. The laser beam again falls exactly onto the section line marked on the skin.
p. Adjustment of the angle β. For this purpose, the cylinder 6 is rotated about its longitudinal axis. The rotation is controlled using the inclinometer 5 provided for this purpose. Thereafter, a sufficiently stiff and long targeting pin is inserted, together with the matching guide sleeve 7, into the work bore 31 of the cylinder 6. The cylinder 6 is advanced, and at the same time the navigation unit is moved on the crossbeam 3 until the tip of the pin touches the place marked in the section line where the insertion axis passes through the skin. With the correctly adjusted angle α, the laser beam then falls exactly on the center of the work bore. (This affords the third possibility of controlling the cylinder tilt, cf. item n.) If only a cannula is needed for the intervention, e.g. for a biopsy or punch biopsy, the cannula can already be used instead of the targeting pin.
q. In this phase of the intervention, a CT section can already be used to determine whether the imaged axis of the targeting pin or cannula lies exactly in the insertion axis. If this is not the case, the angle β and the position of the navigation unit on the crossbeam 3 have to be correspondingly readjusted. Each new adjustment is controlled with a CT section.
r. Skin incision at the insertion site.
s. The targeting instrument is introduced through the guide sleeve 7 as far as the target organ 44. Control with a CT section. If necessary, correction of the position of the targeting instrument, as described under item q.
t. With the targeting instrument in the correct position, the biopsy or instillation is performed. In this case, completion of the intervention.
u. If another intervention, e.g. the implantation of a pin or screw is intended, the targeting instrument is removed. The instruments necessary for the intervention (e.g. drill, biopsy punch) and/or implants (e.g. screws, pins) are introduced through the matching guide sleeves 7 into the target organ 44. The individual steps of the intervention can be controlled using CT sections.
v. For the actions described under items t. and u., before an instrument or implant (object) is introduced into the target organ 44, it is necessary to measure the depth to which the object is to penetrate into the latter. To permit this, the instruments are equipped with a scale whose position is adapted to the length of the matching guide sleeve (cf. FIG. 9). As is shown in FIG. 10, with the aid of this scale, the instrument or implant can be inserted exactly to the predetermined depth/position (cf. FIG. 2).

In the case of an insertion axis inclined mainly toward the horizontal, the navigation unit 30 provided for this purpose is secured on a vertical support column 2. The CT navigation takes place in principle according to the CT navigation sequence described from item a. to item u.

Interventions on bones may require particularly effective fixing of the target area. This fixing takes place before the preparation of the scout image. Particularly suitable means for this fixing are pins or screws which are anchored near the target area on the bone, are secured with the aid of special devices on the base plate 1 or on the gantry, and are removed again after completion of the intervention. The implantation of the fixing implants takes place in the manner described above from items 1 to 21.

In the CT navigation described, the gantry 41 or the support rails always remain outside the gantry only the working end of the cylinder 6 should be located inside the gantry. If the gantry has to be tilted toward the patient's head, the targeting appliance is therefore preferably placed toward the feet relative to the gantry. The targeting appliance is placed toward the head from the gantry if the gantry has to be tilted toward the feet.

The maximum angle α, i.e. the maximum inclination of the work plane from the vertical, depends on the tiltability of the gantry. If this is in each case 30 degrees toward the head and toward the feet, a total range of 60 degrees is available for the CT navigation. The tilting of the object 42 or of the instrument/implant, i.e. in the sense of the angle β, is theoretically not limited.

A number of details are explained again in greater detail below with reference to the drawings:

In FIG. 1, the preparation for a CT navigation, taking as an example a translaminar facet screw connection on the lumbar vertebral column, is shown in the lateral scout image. The plotted anatomical marks d are determinant for the tilting of the gantry about the angle α from the vertical a. These marks define where the implant axis b (FIG. 2) lying in the plane c passes through the spinous process and the emergence point of this axis on the base of the transverse process. The appropriate section plane (work plane) c is selected from a series of test sections b on the basis of the section images shown on the screen.

The section image or work plane selected from the test sections b is shown in FIG. 2. In the translaminar facet screw connection, the screw lying in the work plane, as indicated by the line b (insertion axis), must pass through the dorsal vertebral elements with the facet articulations 44a. The insertion axis encloses, together with the vertical a lying in the plane, the angle β. The insertion axis intersects the line (section line of the work plane with the skin) plotted at the skin level c (FIGS. 2 and 7) at a distance d from the wire e affixed to the skin (cf. FIGS. 2 and 7). At this location, the objects 42 or the instruments and implant are introduced through a skin incision at the angle β of the insertion axis. The length and penetration depth f of an instrument or implant can be determined on the basis of the section image.

FIG. 3 shows the targeting device without devices for securing the targeting appliance to the CT table. This is a variant with vertical support columns 2, which can extend in the manner of a telescope, and with a rotatable crossbeam 3′. The vertically oriented support columns 2′, extendable in the manner of a telescope, are displaceable on the base rails 12 on the base plate 1. The navigation unit 30′ mounted on the rotatable crossbeam 3′ comprises a cylinder retainer 4 with an inclinometer 5 for the angle beta. The cylinder 6, which does not produce a shadow in radiological terms, is mounted such that it can be displaced in and also fixed in the cylinder retainer 4, a guide sleeve 7 is inserted in the work bore 31 of the cylinder 6, and the object 42, for example an instrument, can be inserted into the guide sleeve 7. Provided on the base plate 1 there are displaceable lateral struts 9 on which the belts 10 stretched across the patient's body are secured. Transverse displacement of the lateral struts 9 on the base plate 1 can take place by means of retaining plates 11. Lateral guide rails 12 are provided on the base plate 1 so that the whole targeting device can be moved in the manner of a slide on the base rails 12 along the side edges of the base plate 1. An inclinometer 14 for measuring the angle α is mounted on the crossbeam 3′. To permit good mobility of the whole targeting device 43 on the base plate 1, guide elements 16 in the form of guide elements are also provided. For controlling the angle α, the pointer 17 is fitted on the cylinder retainer 4, and the marking lines 18 are arranged on the crossbeam 3′.

A navigation unit 30 is shown in FIG. 4, illustrating the cylinder retainer 4, the inclinometer 5 and the cylinder 6. A sleeve 7 is inserted in the work bore 31 of the cylinder 6, and an instrument 42 is inserted in the sleeve 7. The pointer 17 is fitted on the cylinder retainer 4.

FIG. 5 shows in particular the base plate 1 with various fixtures. Belts 8 for securing the base plate 1 in the longitudinal direction on the displaceable part of the CT table can be seen here. Displaceable lateral struts 9 on the base plate 1 can also be seen, and also belts 10 that can stretch across the patient's body. The retaining plates 11 for transverse displacement of the lateral struts 9 on the base plate 1 can also be seen again here. FIG. 5 also shows the lateral base rails 12 and, in addition, a device 13 for securing the base plate 1 in the direction transverse to the displaceable part of the CT table.

FIG. 6 shows the gantry 41 with a navigation unit 30 mounted on a vertical support column 2. The gantry 41 is formed by the vertical support columns 2 and the crossbeam 3. Here, the two-part cylinder retainer 4 is provided with a hinge, a part 4a being displaceable on the support column 2 and a part 4b being rotatable about the axis 15. The figure also shows the cylinder 6, and the sleeve fitted in the work bore 31 of the cylinder and carrying the instrument 42. In this arrangement, the devices 16 are again particularly evident, which form guide elements for the slide-like displacement of the CT targeting appliance on the base rails 12. An inclinometer 19 that can be turned through 90 degrees is also arranged here for the angles α and

FIG. 7 is a schematic representation of the CT navigation principle according to the invention (cf. FIGS. 1 and 2). The CT section plane into which the instrument/implant (object) is to be brought (work plane c in FIG. 1) is determined in the lateral CT scout image (FIG. 1). It is tilted about the angle alpha into the work plane. The targeting instrument (large arrow) is maneuvered with the targeting appliance into the work plane and, in the latter, is maneuvered into the predetermined implantation axis (b in FIG. 2).

Determination of the point of intersection of the implantation axis with the skin: The laser light (small arrows) emitted in the work plane from the gantry marks the section line c of the work plane with the body surface. The distance d between the wires (e) affixed to the skin and the point visible in the CT image where the implantation axis passes through the skin (d in FIG. 2) is marked on the skin. The targeting instrument already set at the angle b (cf. FIG. 2) in the CT targeting appliance is moved along the line c until it touches the location of the passage. When the targeting instrument lies exactly in the axis b (FIG. 2) in the CT control, the instrument can be introduced into the body.

The letters in FIG. 7 show the following:

G—gantry; small arrows—laser light emitted from the gantry; large arrow—implantation axis; c—section line of the work plane with the body surface; e—wires affixed to the skin; d—distance of implantation axis from the wire at skin level; a—vertical; b—angle of the implantation axis with respect to the vertical in the work plane (b in FIG. 2).

FIGS. 8a and 8b illustrate the course of the CT navigation according to the invention, where a indicates the tilt angle of the gantry G of the computer tomograph into the work plane c (FIG. 1) and 42 indicates the target organ. According to FIG. 8, all the maneuvers, in particular the adjustment of the targeting appliance and the insertion of the targeting instrument or implant, take place outside the gantry G. According to FIG. 8b, in order to prepare the control images, the CT table with the patient on it is driven into the position of the work plane c stored in the CT.

FIG. 8b shows how, in order to prepare the control images, the CT table with the patient on it is driven into the position of the work plane c stored in the CT.

FIG. 9 shows the guide sleeve 7 with a matching instrument 42 with measurement scale. The distance from the tip of the instrument to the start of the scale corresponds exactly to the length of the guide sleeve.

FIGS. 10a and 10b show an example of how the depth of penetration of an instrument or implant is measured, using the example of percutaneous translaminar fixation of lumbar facet articulations.

FIG. 10a shows how the guide sleeve 7 inserted in the cylinder 6, in the object axis b (cf. FIG. 2), is advanced through the skin incision to the target organ 44 (lumbar vertebra, cf. FIG. 2). When the instrument 42 (drill) inserted in the guide sleeve is applied to the bone, the 0 mark on the scale lies exactly at the rear end of the guide sleeve. The depth of penetration f (cf. FIG. 2) of the drill can then be controlled with the scale.

FIG. 10b shows how the drill is introduced by the length f into the vertebra. The arrow marks on the scale the rear end of the path f.

The implant is inserted in principle in the same way. The guide sleeve 7 remains in the same position. The implant having the length f is implanted by the length f with the aid of a scaled screwdriver.

In the case of a biopsy, the guide sleeve is introduced close to the target object, and the distance of the guide sleeve from the location in the target object from which the biopsy specimen is to be taken is measured. The cannula or biopsy punch matching the guide sleeve is then forced by the distance f into the target object.

### LIST OF REFERENCE NUMBERS

1 base plate

2, 2′ support columns/2a, 2b telescopic parts

3, 3′ crossbeam

4 cylinder retainer/4a, 4b closing parts

5 inclinometer

6 cylinder

7 sleeve

8 belt

9 strut

10 belt

11 retaining plates

12 guide rail

13 device

14 inclinometer

15 axis

16 guide elements

17 pointer

18 marking line

19 inclinometer with push-in spirit level

30 navigation unit

31 work bore

40 targeting appliance

41 gantry

42 implant/instrument

43 targeting device

44 target organ

45 longitudinal axis of cylinder

1. A device for placing instruments or implants in body organs with the aid of a targeting appliance interacting with a computer tomography appliance,
the targeting appliance being arranged in the form of a navigation unit on a support which is displaceable on a substructure, in the manner of a slide, and which can be fixed in relation to a substructure mounted on a CT table,
the navigation unit comprising a retaining element for holding and guiding an instrument an implant or another object,
the navigation unit being able to be displaced and/or rotated in one or more planes in relation to the support and the retaining element being able to be displaced and/or rotated in one or more planes in relation to the navigation unit, and
the navigation unit and the retaining element being able to be mutually fixed an the adopted position, wherein a computer integrated in the CT appliance is able to store at least one position of the CT table, which position corresponds to a work plane determined by test sections,
in that the object, implant or instrument to be inserted can be brought into said work plane with the aid of the targeting appliance and can be further moved only in this plane, and
in that the CT table is displaceable such that it can be moved out from a gantry of the computer tomograph and repeatedly returned according to the stored position of the work plane in such a way that the intervention can be navigated on the basis of updatable CT images.

2. The device as claimed in claim 1, characterized in that the support is equipped with support columns displaceable in the manner of a slide on the base plate and, if appropriate, with an additional crossbeam, and the navigation unit (30) is arranged displaceably and, if appropriate, rotatably on the support columns and/or on the crossbeam but in such a way that it can be fixed.

3. The device as claimed in claim 1, characterized in that the navigation unit comprises a cylinder which is rotatable therein about the longitudinal axis thereof but which can be fixed in relation to it, and which cylinder carries the retaining element comprising a sleeve and provided for receiving and guiding the targeting instrument or the like.

4. The device as claimed in claim 1, characterized in that the retaining element comprises a sleeve having a bore both for use of a drill and also for insertion of holding elements, optionally pins or screws.

5. The device as claimed in claim 3, characterized in that the cylinder has one or more bores or polygonal openings formed in it for receiving the retaining element comprising a sleeve.

6. The device as claimed in claim 3, characterized in that the cylinder is equipped with an angle-measuring appliance.

7. The device as claimed in claim 3, characterized in that the cylinder is made of metal.

8. The device as claimed in claim 3, characterized in that the cylinder is made of radioparent material.

9. The device as claimed in claim 1, characterized in that the support and/or the navigation unit, for controlling the angle settings, have openings and/or markings for adaptation to a laser beam which is emitted from the computer tomograph and which is oriented toward the body of a patient.

10. The device as claimed in claim 1, characterized in that the support is arranged on, and can be fixed in relation to, a base plate which is able to be fixed on the computer tomograph table and which has laterally arranged base rails.

11. The device as claimed in claim 10, characterized in that the base plate is made of a material that allows X-rays to pass through.

12. The device as claimed in claim 10, characterized in that belts which can be stretched transversely across a patient's body are fixed on the side edges of the base plate.

13. The device as claimed in claim 10, characterized in that the base plate can be secured on the computer tomograph table by belts.

14. The device as claimed in claim 1, characterized in that the support comprises an arc-shaped support rail and can be rotated together with the navigation unit the section plane fixed by the computer tomograph and can be fixed in this position relative to the base plate.

15. A method for operating the device as claimed in claim 1, characterized by the following steps:
a) preparing test sections through the target region with the computer tomograph,
b) determining a work plane on the basis of section images of the test sections,
c) adjusting the gantry to the level of the work plane,
d) storing the position of the computer table at which the latter is adjusted to the level of the work plane,
e) carrying out the intervention, with navigation being carried out on the basis of updated CT images.
